(19) United States
(12) Patent Application Publication
(10) Pub. No.: US 2016/0174857 A1
(43) Pub. Date: Jun. 23, 2016

(54) Wearable Apparatus, System and Method for Detection of Cardiac Arrest and Alerting Emergency Response

(71) Applicant: Eggers & Associates, Inc., Dublin, OH (US)

(72) Inventors: Philip E. Eggers, Dublin, OH (US); Eric A. Eggers, Portland, OR (US); Benjamin Z. Bailey, Athens, OH (US)

(73) Assignee: Eggers & Associates, Inc., Dublin, OH (US)

(21) Appl. No.: 14/970,801

(22) Filed: Dec. 16, 2015

Related U.S. Application Data
(60) Provisional application No. 62/095,239, filed on Dec. 22, 2014.

(51) Int. Cl. A61B 5/024; A61B 5/11; A61B 5/053; A61B 5/021; A61B 5/00; A61B 5/01

(52) U.S. Cl. CPC A61B 5/02438; A61B 5/0022; A61B 5/01; A61B 5/0531; A61B 5/021; A61B 5/681; A61B 5/7282; A61B 5/746; A61B 5/1112

(57) ABSTRACT

The disclosure provides a wearable cardiac arrest detection and alerting device that incorporates a non-invasive sensor based on optical and/or electrical signals transmitted into and received from human tissue containing blood vessels, and that transcutaneously quantifies the wearer's heart rate. The heart-rate quantification enables the detection of the absence of any heart beat by the wearable detection and alerting device indicative of the occurrence of a cardiac arrest, wherein the heart is no longer achieving effective blood circulation in the individual wearing the device. The display on the wearable cardiac arrest detection and alerting device may include the elapsed time since the time of detection of a heart rate that is below a predetermine lower limit value, i.e., the detected occurrence of a cardiac arrest event.

### CROSS-REFERENCE TO RELATED APPLICATIONS

This application claims benefit of provisional application Ser. No. 62/095,239 filed on Dec. 22, 2014.

### FIELD

The field of this disclosure is an apparatus, system, and method for the detection of the occurrence of cardiac arrest in a human subject followed by the prompt issuance of an audible alarm, as well as a vibration alert and cellular phone transmission of synthesized speech alerts and location of human subject to pre-determined list or alternative list of phone numbers according to a precise Global Positioning Satellite (GPS) derived location of subject. The verbal alerts would be issued to one or more persons and emergency medical services that are capable of providing life saving interventions (referred to hereinafter as “first responders”).

### BACKGROUND

Cardiac arrest, also known as cardiopulmonary arrest or circulatory arrest, is a sudden stop in effective blood circulation due to failure of the heart to contract effectively or at all. Medical personnel may refer to an unexpected cardiac arrest as a “sudden cardiac arrest” (SCA).

A cardiac arrest is different from, but may be caused by, a heart attack, where blood flow to the muscle of the heart is impaired. It is different from congestive heart failure wherein the blood circulation level is below normal, but the heart is still pumping sufficient blood to sustain life. It is known that a number of risk factors can contribute to one of the principal causes of cardiac arrest, viz., a delayed repolarization of the heart following a heart beat, an effect known as the Long QT Syndrome. Risk factors for the Long QT Syndrome include, for example, liver or renal impairment, family history of Long QT Syndrome, pre-existing cardiovascular disease, electrolyte imbalance, and interacting drugs such as common antibiotics.

Arrested blood circulation associated with cardiac arrest prevents delivery of oxygen and glucose to the body. The lack of oxygen and glucose to the brain is associated with a loss of consciousness and abnormal or absent breathing. Brain injury is likely to occur if cardiac arrest goes untreated for more than about four to five minutes. It is widely known that the chance of survival decreases about 10% for each minute that arrested blood circulation persists. The best chance of survival and neurological recovery requires prompt and decisive treatment to restore the circulation of blood and glucose to the brain, as well as other organs. Unfortunately, the average elapsed time from the moment that a call is placed to a medical emergency service (e.g., service often associated with closest fire station to individual experiencing a cardiac arrest) to the time of their arrival to treat the individual who has experienced a cardiac arrest is about 8 to 10 minutes. A delay of 8 to 10 minutes until emergency medical personnel arrive and initiation of cardiopulmonary resuscitation (CPR) and/or external defibrillation following cardiac arrest most often results in death or severe morbidity of individual who has experienced a cardiac arrest.

Sudden cardiac death (SCD) accounts for about 15% of all deaths in Western countries with a total of 330,000 deaths per year in the United States. The lifetime risk of sudden cardiac death in the U.S. is about 17% and is three times greater in men than in women. However, beyond the age of 85, this gender difference in sudden cardiac deaths disappears.

The most effective treatment for cardiac arrest is the immediate application of electrical current to the chest region containing the heart, a procedure known as defibrillation. Cardiopulmonary resuscitation (CPR) is used alone or in combination with defibrillation to provide circulatory support and/or to induce an effective heart rhythm. In the past, defibrillator devices have been only used by trained emergency response personnel who arrived at the location of the individual who suffered a cardiac arrest, as well as medical staff if the cardiac arrest has occurred while the individual is in the hospital or skilled nursing care facility.

However, automated versions of a defibrillator, known as an “automated external defibrillator” (AED) are now widely available. An AED is a portable electronic device that automatically diagnoses the life-threatening cardiac arrhythmias of ventricular fibrillation and ventricular tachycardia in a subject and is able to treat them through defibrillation, the application of electrical therapy which restarts the heart function and/or stops the arrhythmia, allowing the heart to re-establish an effective rhythm to enable essential circulation of blood to the brain and other organs. With simple audio and visual commands, newer versions of AEDs are designed to be simple enough for use by a layperson and the use of AEDs is taught in first aid, certified first responder, and basic life support level CPR classes. Also, the newer versions of AEDs manufactured since 2003 utilize biphasic algorithms that produce two sequential lower-energy shocks of 120 to 200 joules, with each shock moving in an opposite current-flow direction between externally applied electrode pads. This lower-energy waveform has been clinically proven to be more effective in re-establishing an effective heart rhythm, as well as offering a reduced rate of complications and reduced recovery time. Some of the latest versions of AEDs have received allowance by the Food and Drug Administration (FDA) for purchase directly by the public in the U.S. without a prescription or without initial purchase by qualified medical personnel. For example, a complete portable AED system manufactured by Phillips (Phillips HeartStart Home Defibrillator) is available online from Amazon at a price of about $1,100 based on pricing in 2015.

Automated external defibrillators now are easy enough to use that most states in the United States include the “good faith” use of an AED by any person under the Good Samaritan laws. “Good faith” protection under a Good Samaritan law means that a volunteer responder (not acting as a part of one's occupation) cannot be held civilly liable for the harm or death of a victim by providing improper or inadequate care, given that the harm or death was not intentional and the responder was acting within the limits of their training and in good faith. In the United States, Good Samaritan laws provide some protection for the use of AEDs by trained and untrained responders. In addition to Good Samaritan laws, Ontario, Canada also has the Chase McEachern Act (Heart Defibrillator Civil Liability) that passed in June, 2007 that protects individuals from liability for damages that may occur from their use of an AED to save someone's life at the immediate scene of an emergency unless damages are caused by gross negligence.

Although widely available CPR training and fully automated AED technology now exists to provide for prompt intervention when an individual suffers a cardiac arrest and becomes unconscious, there remains an unmet need to alert family member(s), neighbor(s), office workers, assisted-living or skilled nursing facility staff, and emergency services at the moment a cardiac arrest has occurred. By way of example, someone may be in his or her office at a place of employment and behind a closed door when a cardiac arrest has occurred. As a result, even though an AED device may be present in the office and co-workers trained in its use, as well as the performance of CPR, no one in the office may become aware during the first critical minutes following the onset of a cardiac arrest, thereby leading to sudden cardiac death. This same situation can occur in many other settings, including, for example, the home, hotel, assisted-living facility, skilled-nursing facility, or other settings where life preserving intervention is immediately available, if potential responders can be alerted to the occurrence of a cardiac arrest in their midst. In addition, many elderly individuals live alone well into their 80's and some even into their 90's, so the need to alert potential responders in their neighborhood, as well as emergency services via a 911 call, is even more critical in the event the occurrence of a cardiac arrest.

The present disclosure overcomes the critical need to immediately alert potential first responders (e.g., family member(s), co-workers, fitness facility staff, neighbor(s), assisted living facility staff, hotel staff, or any individual with an application on their smart phone or smart device that informs them of a cardiac arrest event and its location) prior to the arrival of professional emergency medical services by detecting that a cardiac arrest has occurred, immediately issuing an audible alarm, and then dialing pre-established phone numbers to alert potential first responders with [a] the individual's name, [b] individual's exact location including GPS-derived latitude and longitude coordinates, [c] time of occurrence of cardiac arrest and, optionally, [d] the location of nearest AED device(s) in the event the nearest AED device(s) is(are) geocoded into a data base accessible by a server. The term “server”, as used herein, refers to single-purpose and specially developed computer program(s) and computer hardware operating at a physical location different from the location of the wearable cardiac arrest detection and alerting device and that the server is accessible to wearable cardiac arrest detection and alerting device via cellular telephone communication. The server waits for transmitted data and an alert from a wearable cardiac arrest detection and alerting device or accessory cellular phone and programmable device and, once data and alert are received, responds by utilizing a programmed protocol and accessible data bases to identify and issue a synthesized voice alert to identified first responders including professional emergency medical service providers (e.g., providers accessible via call to 911 in the U.S.).

In addition, an application or applications (hereinafter referred to as an “App” or “Apps”) may be installed in the smart phone or other smart device of “first responder” volunteers that could inform them that an individual has suffered a cardiac arrest and the individual's precise location. This process could provide a much broader pool of potential first responders by expanding the set of potential candidates who would be in close proximity to someone who has suffered a cardiac arrest and could provide the most prompt intervention. This would expand smart phone applications (i.e., Apps) from widely used “social media” participation into “social lifesaving” participation. To further enable any potential first responders to provide the most effective level of intervention for an individual suffering a cardiac arrest, AED device(s), whether in the in home of the individual suffering a cardiac arrest or in a nearby location, could be geocoded such that the location of the nearest one or more AED device(s) would be accessible in the server data base. The server would then communicate the location of the nearest known (i.e., geocoded) AED device(s). This would enable a potential first responder who arrives at the location of the individual suffering a cardiac arrest to promptly access the nearest AED device and provide the most effective intervention. In the present disclosure, “first responders” refers to those individuals who can potentially intervene with life saving CPR and/or external defibrillation prior to the arrival of emergency medical services summoned through a telephone call to an emergency phone number (e.g., such as 911 in the U.S.).

### BRIEF SUMMARY

The apparatus, system, and method of the present disclosure utilizes a wearable cardiac arrest detection and alerting device that incorporates a non-invasive sensor, based on optical and/or electrical signals transmitted into and received from human tissue containing one or more blood vessels, that transcutaneously quantifies the wearer's heart rate. The heart-rate quantification enables the detection of the absence of any heart beat by the wearable detection and alerting device indicative of the occurrence of a cardiac arrest wherein the heart is no longer achieving effective blood circulation in the individual wearing the device.

The display on the wearable cardiac arrest detection and alerting device may advantageously include the elapsed time (e.g., display of elapsed minutes and seconds) since the time of detection of a heart rate that is below a predetermine lower limit value, i.e., the detected occurrence of a cardiac arrest event. The elapsed time since the detected occurrence of a cardiac arrest event would inform the one or more first responders of the duration since the occurrence of the cardiac arrest event.

By way of example, but without limitation, the apparatus, system, and method of a first embodiment of the present disclosure for the detection and alerting of first responders in the event of a cardiac arrest is a wearable cardiac arrest detection and alerting device, such as a wristwatch device or bracelet, that includes [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting device to reduce noise and increase signal-to-noise ratio of signals used to continuously derive an accurate heart rate value, [e] algorithm to continuously analyze measured photon signals to determine whether the measured photon signals are within a predetermined range to confirm that wearable cardiac arrest detection and alerting device is properly functioning and is properly positioned on the individual being monitored and, if measured photon signal levels are within a pre-determined range, continuously derive heart rate value, [f] algorithm to continuously analyze measured heart rate values to determine whether a cardiac arrest has occurred or is imminent, [g] audible alarm in the event that a cardiac arrest has occurred or is imminent, [h] global positioning satellite (GPS) based receiver or equivalent position locating component to determine latitude and longitude of wearable cardiac arrest detection and alerting device, [i] look-up table in software to determine whether wearable cardiac arrest detection and alerting device is at any of the pre-programmed locations frequented by the individual being monitored by the wearable cardiac arrest detection and alerting device (e.g., locations, such as, for example, individual's home, another home, office, fitness facility, or thelike), [j] cellular phone communication component typical of widely used cell phones to place calls in the event a cardiac arrest has occurred or is imminent to a pre-programmed, pre-established list of phone numbers including 911 (for use in the U.S.) or other medical emergency response phone number and any other first responders associated with a pre-programmed locations frequented by the individual being monitored by the wearable cardiac arrest detection and alerting device in the event the wearable cardiac arrest detection and alerting device is determined to be at one of the pre-programmed locations, [k] audible synthesized speech used in issued phone calls to annunciate occurrence of a cardiac arrest, identify the individual's name and specify the exact location of the individual in the form of his or her GPS or equivalent device derived coordinates and, if the individual is at a location with pre-established GPS or equivalent device derived coordinates, the actual address of the individual, and [I] wireless or direct connection to wearable cardiac arrest detection and alerting device from external device (e.g., cell phone) to add look-up table of locations and associated phone numbers corresponding to detected latitude and longitude of wearable cardiac arrest detection and alerting device at time of occurrence of cardiac arrest or imminent cardiac arrest.

By way of example, but without limitation, the apparatus, system, and method of a second embodiment of the present disclosure for the detection and alerting of first responders in the event of a cardiac arrest is a combination of both [a] a wearable cardiac arrest detection and alerting device, such as a wristwatch device or bracelet and [b] an accessory cellular phone and programmable device maintained within the proximity of the wearable cardiac arrest detection and alerting device (e.g., the cellular phone and programmable device within 10 to 100 meters of wearable cardiac arrest detection and alerting device) during the period of monitoring. The wearable cardiac arrest detection and alerting device of a second embodiment of the present disclosure includes [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting device to reduce noise and increase signal-to-noise ratio of signals used to continuously derive heart rate value, [e] audible alarm in the event that a cardiac arrest has occurred or is imminent and [f] wireless communication hardware and software (e.g., Bluetooth ultra-high frequency transmitter) to transmit heart-rate values to accessory cellular phone and programmable device. The accessory cellular phone and programmable device includes [a] wireless communication hardware and software (e.g., Bluetooth ultra-high frequency transmitter) to receive heart-rate values from the wearable cardiac arrest detection and alerting device [b] algorithm to continuously analyze measured photon signal data received from the wearable cardiac arrest detection and alerting device to determine whether the measured photon signals are within a predetermined range to confirm that wearable cardiac arrest detection and alerting device is properly functioning and is properly positioned on the individual being monitored and, if measured photon signal levels are within a pre-determined range, continuously derive heart rate value, [c] algorithm to continuously analyze measured heart rate values to determine whether a cardiac arrest has occurred or is imminent, [d] audible alarm in the event that a cardiac arrest has occurred or is imminent, [e] global positioning satellite (GPS) based receiver or equivalent position locating component to determine latitude and longitude of wearable cardiac arrest detection and alerting device, [f] look-up table in software to determine whether wearable cardiac arrest detection and alerting device is at any of the pre-programmed locations frequented by the individual being monitored by the wearable cardiac arrest detection and alerting device (e.g., locations such as individual's home, another home, office, fitness facility), [g] cellular phone communication component typical of widely used cell phones with a pre-programmed, pre-established list of phone numbers including 911 (for use in the U.S.) and any first responders associated with a pre-programmed locations frequented by the individual being monitored by the wearable cardiac arrest detection and alerting device in the event the wearable cardiac arrest detection and alerting device is determined to be at one of the pre-programmed locations, and [h] audible synthesized speech to annunciate in placed phone calls the occurrence of a cardiac arrest, identify the individual's name and specify the exact location of the individual in the form of his or her GPS or equivalent device derived coordinates and, if the individual is at a location with pre-established GPS or equivalent device derived coordinates, the actual address of the individual.

By way of example, but without limitation, the apparatus, system, and method of a third embodiment of the present disclosure for the detection and alerting of first responders in the event of a cardiac arrest includes [a] a wearable cardiac arrest detection and alerting device such as a wristwatch device incorporating cellular communication capability and [b] a server that can receive a cellular phone call from the accessory cellular phone and programmable device enabling the server to immediately identify the phone number(s) of the closest first responders based on the GPS derived location of the subject and immediately issues voice-based phone call alerts to the identified closest first responder(s) as well as to identified emergency medical services associated with the country in which the subject is located (e.g., issuing call to 911 if subject is in the U.S.).

The term “server”, as used herein, means a computer program and a machine that waits for an alert via cellular phone communication from a wearable cardiac arrest detection and alerting device or accessory cellular phone and programmable device and responds to the alert according to a pre-programmed set of computer instructions. The pre-programmed set of computer instructions include, by way of example but without limitation, the identification of the phone numbers of the nearest first responder(s) based on the subject's GPS-based location as well as the phone number of the identified emergency medical services associated with the country in which the subject is located (e.g., issuing call to 911 if subject is in the U.S.). The purpose of the server is to share data, hardware and software resources among all subjects using a wearable cardiac arrest detection and alerting device and optional accessory cellular phone and programmable device.

By way of example, but without limitation, the apparatus, system, and method of a fourth embodiment of the present disclosure for the detection and alerting of first responders in the event of a cardiac arrest includes [a] a wearable cardiac arrest detection and alerting device, such as a wristwatch device or bracelet, [b] an accessory cellular phone and programmable device maintained within the proximity of the wearable cardiac arrest detection and alerting device (e.g., the cellular phone and programmable device within 10 to 100 meters of wearable cardiac arrest detection and alerting device) during the period of monitoring, and [c] a server that can receive a cellular phone call from the accessory cellular phone and programmable device with location of accessory cellular phone and programmable device based on the GPS derived location of the subject enabling the server to immediately identify the phone numbers of the closest first responder(s) and immediately issues voice-based phone call alerts to the identified closest first responder(s), as well as to identified emergency medical services associated with the country in which the subject is located (e.g., issuing call to 911 if subject is in the U.S.).

In other embodiments of the present invention, the wearable device transcutaneously measures the blood pressure (in place of or in addition to) heart rate to detect the occurrence of a cardiac arrest in the event the blood pressure decreases below a specified minimum pressure level (e.g., 10 mm Hg).

A rechargeable battery is incorporated in the wearable cardiac arrest detection and alerting device, such as a wristwatch device in the four embodiments of the present disclosure. The rechargeable battery provides the electrical energy required for the various functions performed by the wearable cardiac arrest detection and alerting device for periods of days to weeks between recharging. By way of example, but not limitation, the rechargeable battery may be incorporated into the case of a wristwatch device and/or may be incorporated within the watch band using flexible battery technology or in rigid or flexible form within the links of an expandable watch band.

The digital filtering utilized to minimize signal noise associated with motion artifact may include the use of [a] Moving Average Filtering (in this regard, see Lee, J., et. al., Design of Filter to Reject Motion Artifact of Pulse Oximetry. Comput. Stand. Interfaces 2004; 26: 241-249), [b] Fourier Analysis Filtering (in this regard, see Reddy, K., et. al., Use of Fourier Series Analysis for Motion Artifact Reduction and Data Compression of Photoplethysmographic Signals. IEEE Trans. Instrum. Meas. 2009; 58: 1706-1711), [c] Adaptive Noise Cancellation Filtering using triaxial accelerometer (in this regard, see Asada, H., et. al., Active Noise Cancellation using MEMS accelerometers for Motion-Tolerant Wearable Bio-Sensors. Conf. Proc. IEEE EMBS 2004; 3:2157-2160), [d] Least Mean Square Adaptive Filtering (in this regard, see Wei, P., et. al., A New Wristband Wearable Sensor Using Adaptive Reduction Filter to Reduce Motion Artifact. Proc. of 2008 International Conf. on Information Technology and Applications in BioScience (ITAB, Shenzhen, China). May 2008; 30-31: 278-281 and Ram, M. et. al., A Novel Approach for Artifact Reduction in Photoplethysmographic Signals based on AS-LMS Adaptive Filter. IEEE Instrum, Meas. 2012; 61: 1445-1457), [e] Principal Component Analysis Filtering (in this regard, see Rhee, S., et. al., Artifact-Resistant, Power Efficient Design of Finger-Ring Plethysmographic Sensors. IEEE Trans. Biomed. Eng. 2001; 48: 795-805 and [f] Laguerre Expansion Filtering (in this regard, see Wood, L., et. al., Active Motion Artifact Reduction for Wearable Sensors using Laguerre Expansion and Signal Separation. Proc. IEEE Conference on EMBS Shanghi, China, January 2005; 17-18: 652-655), where all of the above citations are incorporated herein by reference.

The GPS receiver based positioning component relies on electromagnetic wave communication with satellites that orbit the Earth. To determine the exact location of the individual that encounters a cardiac arrest, the GPS receiver within the wearable cardiac arrest detection and alerting device (e.g., wrist watch) or accessory cellular phone and programmable device (typically located within a distance of 10 to 100 meters from the wearable cardiac arrest detection and alerting device) determines the locations of at least three satellites out of a world-wide total of about 24 orbiting satellites above the GPS receiver. The GPS receiver then uses three-dimensional trilateration to determine the exact location of the GPS receiver by mathematically constructing a sphere around each of three satellites that the GPS receiver locates. These three spheres geometrically intersect in two points--on in space, and one on the ground. The point on the ground at which the three spheres geometrically intersect is the exact location of the GPS receiver expressed in units of latitude and longitude on the earth's surface.

The apparatus, system, and method of the present disclosure utilize latitude and longitude coordinate information in two important ways. First, if a location is known in terms of a street address and postal code (e.g., an individuals residence location), the location can be converted into an equivalent set of latitude and longitude coordinates using forward geocoding. For example, one method of forward geocoding is address interpolation. This method makes use of data from a street geographic information system where the street network is already mapped within the geographic coordinate space. Each street segment is attributed with address ranges (e.g., house numbers from one segment to the next). Geocoding takes an address, matches it to a street and specific segment (such as a block, in towns that use the “block” convention). Geocoding then interpolates the position of the address, within the range along the segment, to derive the latitude and longitude coordinates for a specified address. Second, reverse geocoding is utilized to obtain the back (reverse) coding of a point location (latitude and longitude coordinates) into a readable address and place name (if also known). This permits the identification of nearest street address and location name (e.g., hotel name). Utilizing internet-based geocoding services, reverse geocoding enables the conversion of the latitude and longitude coordinates obtained by the GPS component into a readable street address that can be communicated to one or more first responders according to the teachings of the present disclosure. By way of example, GeoNames provides a reverse geocoding web service that is capable of identifying the nearest street address (and place names, if known) from the GPS-derived latitude and longitude coordinates.

The physical addresses and associated phone numbers (e.g., neighbor's phone numbers) of individual's frequented locations or in close proximity to individual's frequented locations (e.g., home address, office address, fitness facility address, hotel(s), airport(s), business addresses) are converted to latitude and longitude coordinates using forward geocoding software available on the internet. The derived latitude and longitude coordinates corresponding to the street addresses and phone numbers are used by wearable cardiac arrest detection and alerting device, accessory cellular phone and programmable device or server to call the phone numbers of identified first responders at the detected address that cardiac arrest occurred as well as to call an emergency medical service (e.g., by placing call to 911 in the U.S.). All issued phone calls include synthesized voice specification of the name of individual experiencing a cardiac arrest and his or her current address. The language used by the voice synthesizer is based on the GPS-derived country in which the wearable cardiac arrest detection and alerting device is located at the time that the individual experiences a cardiac arrest. By way of example, the languages may include, for example, English, Mandarin, Spanish, French, German, Dutch, Italian, Portuguese, Danish, Norwegian, Swedish, Finnish, Russian, Polish, Hungarian, Hindi, Bengali, Javanese, Greek, Arabic, Persian, Japanese, Korean, Vietnamese, and Turkish.

In the event the individual experiencing a cardiac arrest is not at one of the pre-programmed locations and associated phone numbers, the wearable cardiac arrest detection and alerting device, accessory cellular phone and programmable device or server accesses the internet to utilize reverse geocoding thereby converting GPS-derived latitude and longitude coordinates of wearable cardiac arrest detection and alerting device to the nearest physical street address. Once the nearest street address is identified using reverse geocoding, then the wearable cardiac arrest detection and alerting device, accessory cellular phone and programmable device or server accesses the internet to identify phone numbers associated with identified street address. One or more telephone calls are next issued (i.e., in addition to phone call to emergency medical services at, for example 911) to the identified phone number(s) of one or more nearby first responders to alert the one or more first responders that individual at or adjacent to their location has just experienced a cardiac arrest and immediate action is required (e.g., main desk at hotel or restaurant, main number of workplace, front desk of fitness facility, main number of department store or airport).

In addition, an application or applications (hereinafter referred to as an “App” or “Apps”) may be installed in the smart phone or other smart device of “first responder” volunteers that could inform them that an individual has suffered a cardiac arrest and the individual's precise location. This process could provide a much broader pool of potential first responders by expanding the set of potential candidates who would be in close proximity to someone who has suffered a cardiac arrest and could provide the most prompt intervention. This would expand smart phone applications (i.e., Apps) from widely used “social media” participation into “social lifesaving” participation. To further enable any potential first responders to provide the most effective level of intervention for an individual suffering a cardiac arrest, AED device(s), whether in the in home of the individual suffering a cardiac arrest or in a nearby location, could be geocoded such that the location of the nearest one or more AED device(s) would be accessible in the server data base. The server would then communicate the location of the nearest known (i.e., geocoded) AED device(s). This would enable a potential first responder who arrives at the location of the individual suffering a cardiac arrest to access the nearest AED device and provide the most effective intervention.

The audible alarm is combined with synthesized speech to alert first responder that cardiac arrest has occurred and that cardiopulmonary resuscitation (CPR) and external defibrillation, if available, needs to commence immediately. By way of example, but without limitation, upon the detected occurrence of cardiac arrest, the audible alarm emits a tone at a loudness level of, say, 90 decibels at a single or varying frequency interrupted every five seconds to annunciate verbal alert that cardiac arrest has occurred and that (CPR) and external defibrillation (if available) needs to commence immediately.

A detection apparatus, system, and method also are incorporated in the wearable cardiac arrest detection and alerting device, such as a wristwatch type device, in all four embodiments of the present disclosure. By way of example and without limitation, the detection apparatus, system and method may employ one or more of the following methods, such as, for example, [1] sensing of body heat based on direct temperature sensing or indirect infrared temperature sensing, [2] measurement of electrical conductance or impedance of the subject's skin layer adjacent to and in contact with wearable cardiac arrest detection and alerting device, [3] measurement of electrical capacitance of subject's body adjacent to and in contact with wearable cardiac arrest detection and alerting device, and/or [4] mechanical switch or pneumatic switch (e.g., dome switch). One or more of these methods and measured parameters are compared with pre-determined values to determine whether the contact between the wearable cardiac arrest detection and alerting device and the surface individual's body (e.g., wrist) is sufficient to enable heart rate measurement.

Also, in order to further minimize the possibility of issuing a false alarm to first responders, the wearable cardiac arrest detection and alerting device and/or accessory cellular phone and programmable device of the present disclosure will issue an audible alert in the immediate surroundings of the subject, as well as a vibration alert, for a period sufficiently long to enable subject to cancel any false detection of a cardiac arrest prior to the broadcast of an alarm to first responders. By way of example, a distinct 90 dB audible tone would be issued by the wearable cardiac arrest detection and alerting device and/or accessory cellular phone and programmable device for a period of 15 to 30 seconds to enable subject to cancel a false alarm before the apparatus and system of the present disclosure broadcasts the detected occurrence of a cardiac arrest to first responders. A “cancel alarm” function would be incorporated in the wearable cardiac arrest detection and alerting device (i.e., device that is being worn or intended to be worn) and/or in the accessory cellular phone and programmable device so that the subject can prevent the broadcast of any false alarm. By way of example, a false alarm may be caused by wrist-worn wearable cardiac arrest detection and alerting device losing adequate contact with skin surface to enable measurement of the heart rate or the interface between the wearable cardiac arrest detection and alerting device and the subject becoming sufficiently wet to affect the heart rate measurement.

Once the first responders arrive at the location of the individual that is in a state of cardiac arrest, CPR and/or defibrillation using an AED, if readily accessible, would promptly commence while awaiting the arrival of trained emergency medical personnel alerted via the automated 911 call and GPS-based locator for the individual that is in a state of cardiac arrest.

In yet another embodiment of the present disclosure, the wearable cardiac arrest detection and alerting device may be worn at some other location on the human body, by way of example, around the torso, around the upper arm, on a finger in a form similar to a ring or around the head in the form of a head band mounted device.

Other objects of the disclosure will, in part, be obvious and will, in part, appear hereinafter. The disclosure, accordingly, includes the apparatus, system and method possessing the construction, combination of elements, arrangement of parts and steps, which are exemplified in the following detailed description.

### BRIEF DESCRIPTION OF THE DRAWINGS

For a fuller understanding of the nature and advantages of the present method and process, reference should be had to the following detailed description taken in connection with the accompanying drawings, in which:

FIG. 1 is a pictorial representation of a top view of the wearable cardiac arrest detection and alerting device for all four embodiments of the present disclosure;

FIG. 2 is a pictorial representation of a side view of the wearable cardiac arrest detection and alerting device for all four embodiments of the present disclosure;

FIG. 3 is an isometric pictorial representation of a back view of the wearable cardiac arrest detection and alerting device for all four embodiments of the present disclosure showing the heart-rate measuring sensors and magnetic coupling components;

FIG. 4 is an isometric pictorial representation of a back view of the wearable cardiac arrest detection and alerting device for all four embodiments of the present disclosure showing the recharging module positioned over the backside of a wristwatch styled device;

FIG. 5 is a pictorial representation of a top view of the system comprising a wearable cardiac arrest detection and alerting device and server in a first embodiment of the present disclosure;

FIG. 6 is a pictorial representation of a top view of the system comprising a wearable cardiac arrest detection and alerting device and accessory cellular phone and programmable device in a second embodiment of the present disclosure;

FIGS. 7A and 7B combine as labeled thereon to provide a flow chart describing the operation and use of the wearable cardiac arrest detection and alerting device of a preferred embodiment of the present disclosure as seen in FIGS. 1-4 and 8;

FIG. 8 is a pictorial representation of a top view of the system comprising a wearable cardiac arrest detection and alerting device, server and land-line based telephone and/or cellular phone of one or more first responders in a third and preferred embodiment of the present disclosure; and

FIG. 9 is a pictorial representation of a top view of the system comprising a wearable cardiac arrest detection and alerting device, server and land-line based telephone and/or cellular phone of one or more first responders and accessory cellular phone and programmable device in a fourth embodiment of the present disclosure.

The drawings will be described in further detail below.

### DETAILED DESCRIPTION

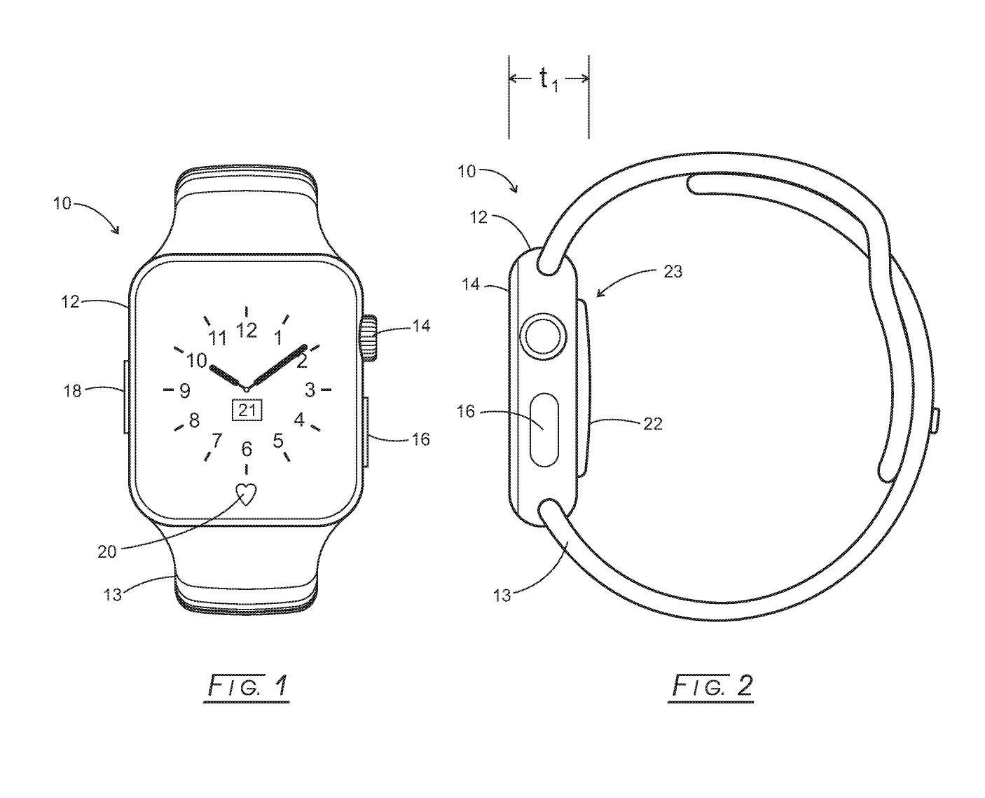

In the disclosusre to follow, initially seen in FIGS. 1 and 2 representing all four embodiments of the present disclosure for the detection and alerting of first responders in the event of a cardiac arrest or imminent cardiac arrest. As seen in the exterior front surface view of a wearable cardiac arrest detection and alerting device, 10, in FIG. 1, wearable cardiac arrest detection and alerting device 10 includes a case, 12, a wrist-band, 13, a clock adjustment stem, 14, an on/off toggle switch for heart rate monitor, 16, a display toggle switch, 18, a heart icon, 20, displayed when heart rate monitoring function is active, and a clock display, 21. As seen in the exterior side view of wearable cardiac arrest detection and alerting device 10 in FIG. 2, a back surface, 23, of wearable cardiac arrest detection and alerting device 10 includes a sensor support member, 22, and sensor, as well as battery charging components (not shown in FIG. 2).

Still referring to FIGS. 1 and 2, wearable cardiac arrest detection and alerting device 10 includes a number of internal components (not seen in FIGS. 1 and 2) including by way of example, but not limited to, [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting to reduce noise and increase signal-to-noise ratio of signals used to continuously derive heart rate value, [e] algorithm to continuously analyze measured photon signals to determine whether the measured photon signals are within a predetermined range to confirm that wearable cardiac arrest detection and alerting is properly functioning and is properly positioned on the individual being monitored and, if measured photon signal levels are within a pre-determined range, continuously derive heart rate value, [f] algorithm to continuously analyze measured heart rate values to determine whether a cardiac arrest has occurred or is imminent, [g] activatable audible alarm, as well as a vibration alert, in the event that a cardiac arrest has occurred or is imminent, [h] global positioning satellite (GPS) based receiver or equivalent position locating component to determine latitude and longitude of wearable cardiac arrest detection and alerting, [i] look-up table in software to determine whether wearable cardiac arrest detection and alerting is at any of the pre-programmed locations frequented by the individual being monitored by the wearable cardiac arrest detection and alerting (e.g., locations such as individual's home, another home, office, fitness facility), [j] cellular phone communication component typical of widely used cell phones to place calls in the event a cardiac arrest has occurred or is imminent to a pre-programmed, pre-established list of phone numbers including 911 (for use in the U.S.) or other medical emergency response phone number and any other first responders associated with a pre-programmed locations frequented by the individual being monitored by the wearable cardiac arrest detection and alerting in the event the wearable cardiac arrest detection and alerting is determined to be at one of the pre-programmed locations, and [k] audible synthesized speech for use in placed phone calls to annunciate occurrence of a cardiac arrest, identify the individual's name and specify the exact location of the individual in the form of his or her GPS or equivalent device derived coordinates and, if the individual is at a location with pre-established GPS or equivalent device derived coordinates, the actual address of the individual.

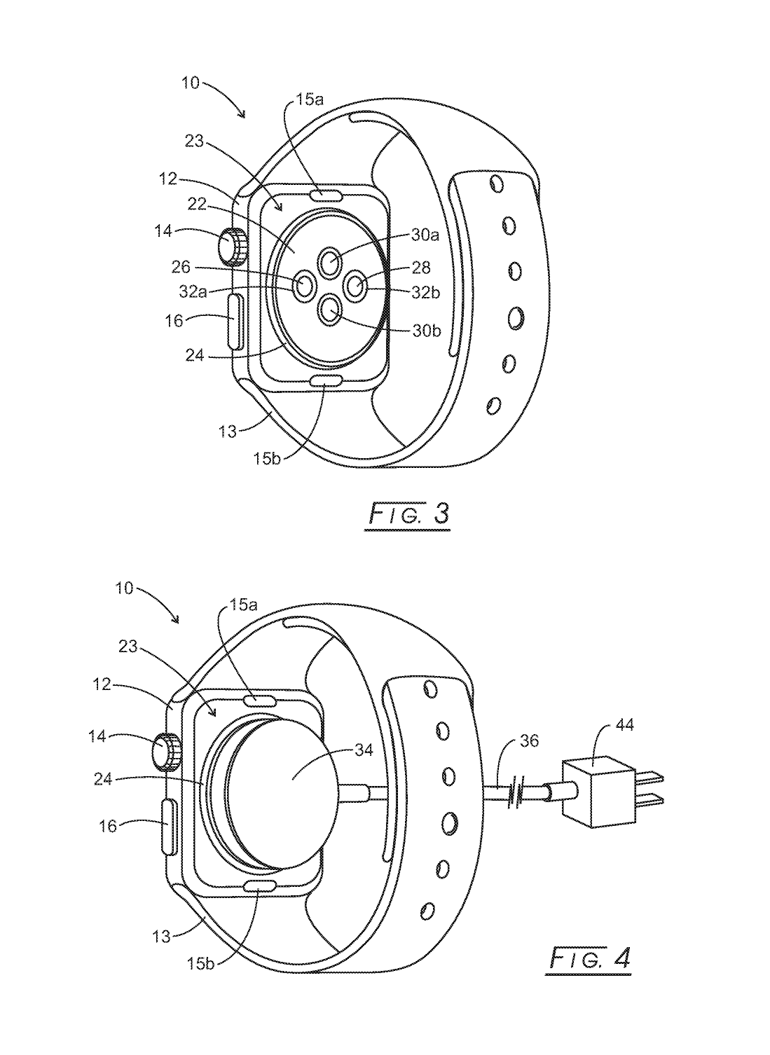

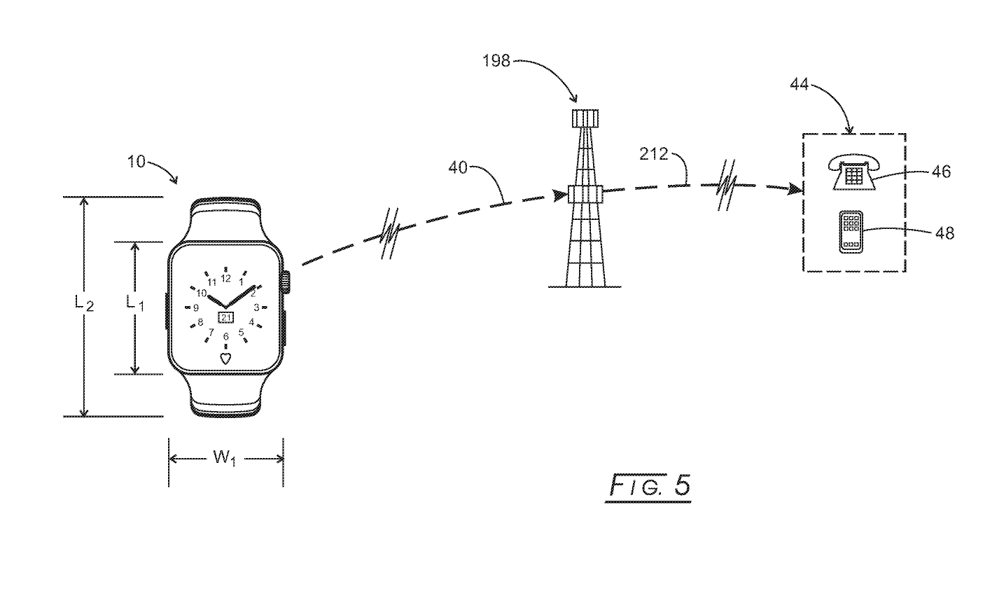

Referring now to FIG. 3, a perspective view of back surface 23 of wearable cardiac arrest detection and alerting device 10 is seen, which includes wrist band release springs, 15a and 15b, a sensor support member, 22, a water-proof sealing gasket, 24, a photon source, 26, of first wavelength Lambdal, a photon source, 28, of second wavelength Lambda2, electro-optical photodetectors, 30a, and 30, and battery charging terminals, 32a and 32b, for coupling to inductive battery charging pod (not shown in FIG. 3). Photon sources 26 and 28 preferably are light emitting diode (LED) components due to their small size and capability to be cyclically energized for very brief periods for energized durations on the order of microseconds to milliseconds.

First wavelength Lambdal may be in the visible red spectrum between 600 nm and 760 nm and second wavelength Lambda2 may be in the infrared spectrum between 800 nm and 950 nm. Alternatively, first wavelength Lambdal may be in the visible green spectrum with a wavelength of 560 nm and second wavelength Lambda2 may be in the visible green spectrum with a wavelength of 577 nm. The two wavelengths in the visible green spectrum are used since the biggest difference in hemoglobin extinction coefficients between deoxyhemoglobin, RHb, and oxyhemoglobin, HbO₂, occur at these two green wavelengths (in this regard, see U.S. Pat. No. 5,830,137, incorporated herein by reference).

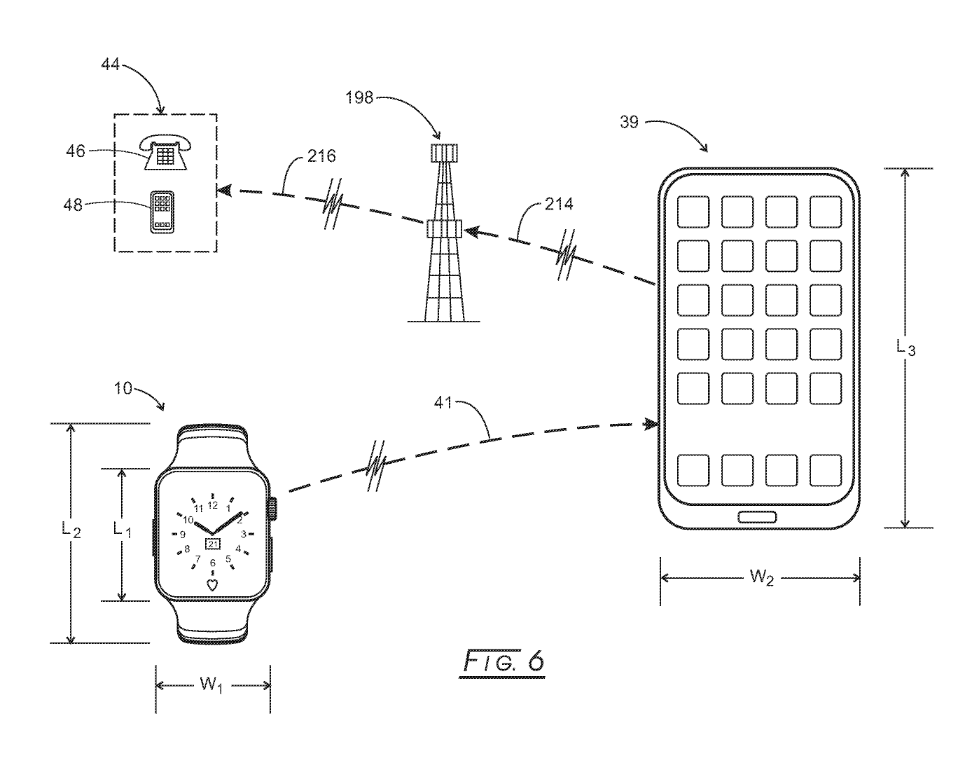

Referring now to FIG. 4, a perspective view of back surface 23 of wearable cardiac arrest detection and alerting device 10 is seen in combination with an inductive battery charging pod, 34, a charging pod cable, 36, and a power source, 44, for inductive battery charging pod 34. Battery charging terminals 32a and 32b seen in FIG. 3 for coupling to inductive battery charging pod 34 may advantageously incorporate a ferromagnetic metal to enable magnetic coupling, optimum alignment and securing of inductive battery charging pod 34 in position adjacent to battery charging terminals 32a and 32b. The magnetic coupling may be achieved with inductive battery charging pod 34 by incorporating one or more permanent magnets within inductive battery charging pod 34 (not seen in FIG. 4), such as, for example, disc shaped neodymium-iron-boron magnets having a diameter ranging from 0.12″ to 0.37″ and thickness ranging from 0.06″ to 0.20″.

A pictorial representation of the apparatus and system of a first embodiment of the present disclosure is presented in FIG. 5 for the detection and alerting of first responders in the event of a cardiac arrest the apparatus. As seen in FIG. 5, the first embodiment includes wearable cardiac arrest detection and alerting device 10, where the wearable cardiac arrest detection and alerting device 10 is in wireless communication, 40, to a cellular receiving/transmitting tower, 198. Wearable cardiac arrest detection and alerting device 10 includes a number of internal components (not seen in FIGS. 1, 2, and 5) including by way of example, but not limited to, [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting to reduce noise and increase signal-to-noise ratio of signals used to continuously derive heart rate value, [e] actuatable audible alarm as well as a vibration alert in the event that a cardiac arrest has occurred or is imminent, [f] one or more sensors to confirm the wearable cardiac arrest detection and alerting is in contact with subjects skin and accessible to source of detectable heart beat (e.g., transcutaneous electrical sensor measuring electrical impedance of skin), [g] wireless communication hardware and software, [h] programmed subject name and/or unique identification (e.g., wearable cardiac arrest detection and alerting device phone number), [i] recharging and programming port (e.g., port to enter subject name or other unique identification), [j] GPS-based component to determine latitude and longitude coordinates of wearable cardiac arrest detection and alerting device, [k] display capable of indicating time, heart rate and warning messages regarding adequate contact with subject to enable detection of true heart rate and battery level, [l] on/off button to cancel alarm in the event of a false detection of a cardiac arrest, and [m] audible synthesized speech to annunciate in subsequent placed phone calls that a cardiac arrest has occurred, identify the individual's name and specify the exact location of the individual in the form of his or her GPS or equivalent device derived coordinates and, if the individual is at a location with pre-established GPS or equivalent device derived coordinates, the actual address of the individual. Wearable cardiac arrest detection and alerting device 10 incorporates a software-based look-up table, as well as access to internet-based phone numbers using reverse geocoding to identify locations and associated phone numbers of first responders corresponding to the GPS-detected latitude and longitude of wearable cardiac arrest detection and alerting at time of occurrence of cardiac arrest or imminent cardiac arrest. The communication of an alert in the event of a cardiac arrest the one or more telephone(s,) 46, and/or cellular phone(s), 48, at locations represented by block 44 of first responders is issued from a cellular receiving/transmitting tower, 198, via a wireless communication path, 212.

By way of example, but without limitation, the apparatus and system of a second embodiment of the present disclosure for the detection and alerting of first responders in the event of a cardiac arrest is illustrated pictorially in FIG. 6. As seen in FIG. 6, the apparatus and system of a second embodiment of the present disclosure includes a combination of both [a] wearable cardiac arrest detection and alerting device 10 and [b] accessory cellular phone and programmable device 39 maintained within the proximity of the wearable cardiac arrest detection and alerting device (e.g., cellular phone and programmable device 39 within 10 to 100 meters of wearable cardiac arrest detection and alerting device 10) during the period of monitoring. Wearable cardiac arrest detection and alerting device 10 includes a number of internal components (not seen in FIGS. 1, 2, and 6) including by way of example, but not limited to, [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting device to reduce noise and increase signal-to-noise ratio of signals used to continuously derive heart rate value, [e] actuatable audible alarm as well as a vibration alert in the event that a cardiac arrest has occurred or is imminent, [f] one or more sensors to confirm the wearable cardiac arrest detection and alerting device is in contact with subjects skin and accessible to source of detectable heart beat (e.g., transcutaneous electrical sensor measuring electrical impedance of skin), and [g] wireless communication hardware and software (e.g., Bluetooth ultra-high frequency transmitter) to transmit heart-rate values to accessory cellular phone and programmable device 39.

Still referring to FIG. 6, accessory cellular phone and programmable device 39 includes [a] wireless communication hardware and software (e.g., Bluetooth ultra-high frequency transmitter) to receive heart-rate values from the wearable cardiac arrest detection and alerting device [b] algorithm to continuously analyze measured photon signal data received from the wearable cardiac arrest detection and alerting device to determine whether the measured photon signals are within a predetermined range to confirm that wearable cardiac arrest detection and alerting device is properly functioning and is properly positioned on the individual being monitored and, if measured photon signal levels are within a pre-determined range, continuously derive heart rate value, [c] algorithm to continuously analyze measured heart rate values to determine whether a cardiac arrest has occurred or is imminent, [d] actuatable audible alarm as well as a vibration alert in the event that a cardiac arrest has occurred or is imminent, [e] global positioning satellite (GPS) based receiver or equivalent position locating component to determine latitude and longitude of wearable cardiac arrest detection and alerting device, [f] look-up table in created software to determine whether wearable cardiac arrest detection and alerting device is at any of the pre-programmed locations frequented by the individual being monitored by the wearable cardiac arrest detection and alerting device (e.g., locations such as individual's home, another home, office, fitness facility), [g] cellular phone communication component typical of widely used cell phones with a pre-programmed, pre-established list of phone numbers including 911 (for use in the U.S.) and any first responders associated with a pre-programmed locations frequented by the individual being monitored by the wearable cardiac arrest detection and alerting device in the event the wearable cardiac arrest detection and alerting device is determined to be at one of the pre-programmed locations, and [h] audible synthesized speech to annunciate in placed phone calls that a cardiac arrest has occurred, identify the individual's name and specify the exact location of the individual in the form of his or her GPS or equivalent device derived coordinates and, if the individual is at a location with pre-established GPS or equivalent device derived coordinates, the actual address of the individual. The communication of an alert in the event of a cardiac arrest to one or more telephone(s) 46 and/or cellular phone(s) 48 at one or more locations represented by block 44 of first responders is issued first from accessory cellular phone and programmable device 39 to cellular receiving/transmitting tower 198 via wireless communication path 214 and then from cellular receiving/transmitting tower 198 to one or more telephone(s) 46 and/or one or more cellular phones 48 via wireless communication path 216.

By way of example, but without limitation, the apparatus, system, and method of a third and preferred embodiment of the present disclosure is shown in FIG. 8 for the detection and alerting of first responders in the event of a cardiac arrest and includes [a] wearable cardiac arrest detection and alerting device 10 such as a wristwatch device incorporating cellular communication capability and [b] a server 206 at some other physical location represented by block 204 that can receive a cellular phone call from the wearable cardiac arrest detection and alerting device 10 enabling the server 206 to immediately identify the phone number(s) of the closest first responders based on the GPS derived location of the subject and immediately issues voice-based phone call alerts to the identified closest first responder(s) as well as to identified emergency medical services associated with the country in which the subject is located (e.g., issuing call to 911 if subject is in the U.S.). As seen in FIG. 8, the apparatus and system of a third embodiment of the present disclosure includes, by way of example, a combination of both [a] a wearable cardiac arrest detection and alerting device 10 and [b] a server, 206, at some other physical location represented by block 204. Wearable cardiac arrest detection and alerting device 10 includes a number of internal components (not seen in FIGS. 1, 2, and 8) including by way of example, but not limited to, [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting device to reduce noise and increase signal-to-noise ratio of signals used to continuously derive heart rate value, [e] actuatable audible alarm as well as a vibration alert in the event that a cardiac arrest has occurred or is imminent, [f] one or more sensors to confirm the wearable cardiac arrest detection and alerting device is in contact with subjects skin and accessible to source of detectable heart beat (e.g., transcutaneous electrical sensor measuring electrical impedance of skin), [g] recharging and programming port (e.g., port to enter subject name or other unique identification), [h] GPS-based component to determine latitude and longitude coordinates of wearable cardiac arrest detection and alerting device, [k] display capable of indicating time, heart rate and warning messages regarding adequate contact with subject to enable detection of true heart rate and battery level, [i] on/off button to cancel alarm in the event of a false detection of a cardiac arrest, and [j] wireless communication hardware and software to transmit the GPS location and an alert related to the occurrence of a cardiac arrest by the subject being monitoring by wearable cardiac arrest detection and alerting device 10.

Still referring to FIG. 8, server 206 located at some other physical location represented by block 204 includes [a] wired or wireless communication hardware and software to receive subject's GPS location and from the wearable cardiac arrest detection and alerting device via wireless communication path 200 [b] look-up table in software to determine whether wearable cardiac arrest detection and alerting device is at any of the pre-programmed locations frequented by the particular individual being monitored by the wearable cardiac arrest detection and alerting device (e.g., locations such as individual's home, another home, office, fitness facility), [c] access to reverse geocoding data base to identify nearest phone numbers of potential first responders based on subject's GPS-derived location in the event the subject is not at one the frequented pre-programmed locations, [d] cellular phone communication component to call identified phone numbers of first responders identified above in [b] or [c] either the pre-programmed phone numbers if the subject is confirmed by reverse-geocoding to be at one of the including 911 (for use in the U.S.), and [e] audible synthesized speech to annunciate in placed phone calls that a cardiac arrest has occurred, identify the individual's name and specify the exact location of the individual in the form of subject's GPS location or equivalent device derived coordinates and, using reverse geocoding data base software, the actual address of the individual. The communication of an alert in the event of a cardiac arrest to the one or more telephone(s) 46 and/or cellular phone(s) 48 at one or more locations signified by block 44 of first responders is issued first from wearable cardiac arrest detection and alerting device 10 to a cellular receiving/transmitting tower 198 via wireless communication path 45 and then from cellular receiving/transmitting tower 198 to server 206 via wireless communication path 200. The communication of an alert in the event of a cardiac arrest proceeds from server 206 via wired and/or a wireless path, 201, to one or more telephone(s) 46 and/or cellular phone(s) 48 at one or more locations represented by block 44.

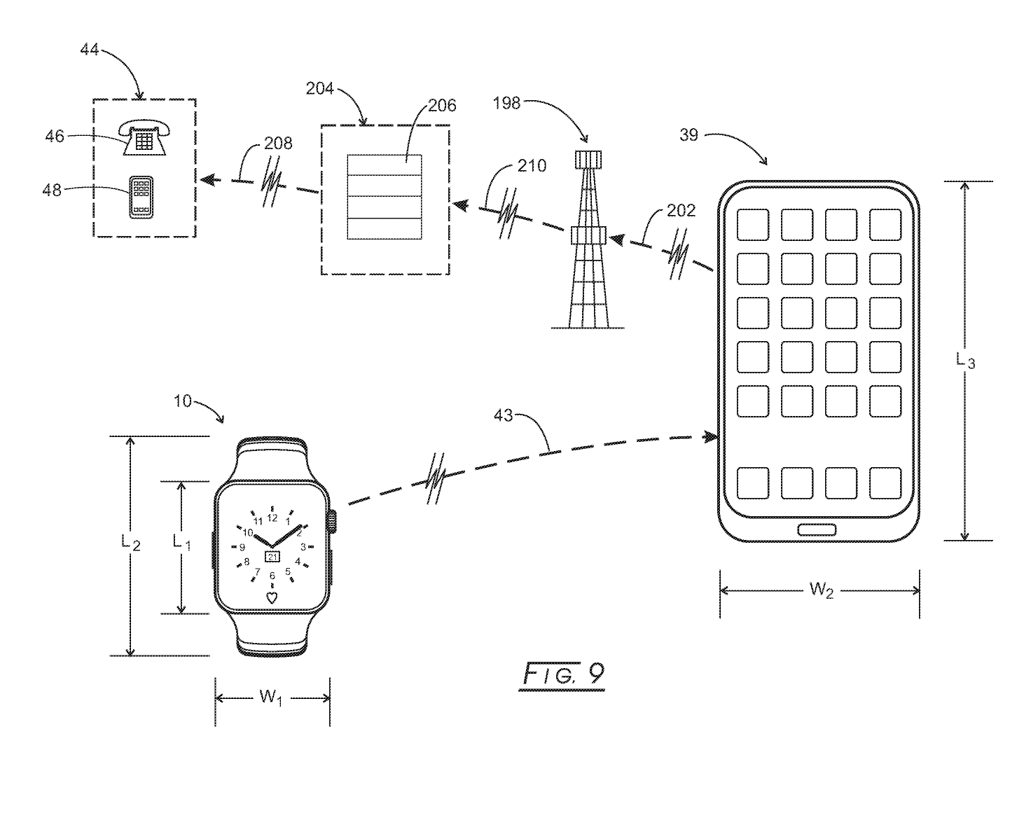

By way of example, but without limitation, the apparatus and system of a fourth embodiment of the present disclosure for the detection and alerting of first responders in the event of a cardiac arrest is illustrated pictorially in FIG. 9. As seen in FIG. 9, the apparatus and system of a fourth embodiment of the present disclosure includes [a] wearable cardiac arrest detection and alerting device 10, [b] accessory cellular phone and programmable device 39 maintained within the proximity of the wearable cardiac arrest detection and alerting device (e.g., cellular phone and programmable device 39 within 10 to 100 meters of wearable cardiac arrest detection and alerting device 10) during the period of monitoring and [c] server 206 at some other physical location represented by block 204. Server 206 is capable of receiving a cellular phone call from accessory cellular phone and programmable device 39 enabling server 206 to immediately identify the phone number(s) of the closest first responders based on the GPS derived location of the subject and immediately issues voice-based phone call alerts to the identified closest first responder(s) as well as to identified emergency medical services associated with the country in which the subject is located (e.g., issuing call to 911 if subject is in the U.S.). Wearable cardiac arrest detection and alerting device 10 includes a number of internal components (not seen in FIGS. 1, 2 and 9) including by way of example, but not limited to, [a] one or more photon sources incorporating one or more electromagnetic energy wavelengths used to continuously or intermittently transmit electromagnetic energy transcutaneously into tissue containing one or more blood vessels, [b] one or more photon detectors to continuously and transcutaneously measure photon signal levels associated with transmitted photons, [c] three-axis accelerometer to generate electrical signal levels corresponding to movement of wearable cardiac arrest detection and alerting device, [d] signal processing hardware componentry and software using photon detector measured electrical signals and accelerometer generated electrical signals to digitally filter artifact caused by movement of the wearable cardiac arrest detection and alerting device to reduce noise and increase signal-to-noise ratio of signals used to continuously derive heart rate value, [e] actuatable audible alarm as well as a vibration alert in the event that a cardiac arrest has occurred or is imminent, [f] sensor to confirm the wearable cardiac arrest detection and alerting device is in contact with subjects skin and accessible to detectable heart beat (e.g., transcutaneous electrical sensor measuring electrical impedance of skin), and [g] wireless communication hardware and software (e.g., Bluetooth ultra-high frequency transmitter) to transmit heart-rate values to accessory cellular phone and programmable device 39.

Still referring to FIG. 9, accessory cellular phone and programmable device 39 includes [a] wireless communication hardware and software (e.g., Bluetooth ultra-high frequency transmitter) to receive heart-rate values from the wearable cardiac arrest detection and alerting device [b] algorithm to continuously analyze measured photon signal data received from the wearable cardiac arrest detection and alerting device to determine whether the measured photon signals are within a predetermined range to confirm that wearable cardiac arrest detection and alerting device is properly functioning and is properly positioned on the individual being monitored and, if measured photon signal levels are within a pre-determined range, continuously derive heart rate value, [c] algorithm to continuously analyze measured heart rate values to determine whether a cardiac arrest has occurred or is imminent, [d] actuatable audible alarm as well as a vibration alert in the event that a cardiac arrest has occurred or is imminent, [e] global positioning satellite (GPS) based receiver or equivalent position locating component to determine latitude and longitude coordinates of accessory cellular phone and programmable device 39, [f] cellular phone communication component typical of widely used cell phones to issue alert to server along with name of individual, other identification (e.g., unique phone number of accessory cellular phone and programmable device 39), and latitude and longitude coordinates of accessory cellular phone and programmable device 39. The communication of an alert in the event of a cardiac arrest to one or more telephone(s) 46 and/or cellular phone(s) 48 at one or more locations represented by block 44 of first responders is issued first from accessory cellular phone and programmable device 39 to cellular receiving/transmitting tower 198 via wireless communication path 202, then from cellular receiving/transmitting tower 198 to server 206 represented at block 204 via wireless path 210 and finally to one or more telephone(s) 46, and/or one or more cellular phones 48 via wireless communication path 208.

Still referring to FIG. 9, server 206 located at some other physical location represented by block 204 includes [a] wired or wireless communication hardware and software to receive subject's GPS location and from the wearable cardiac arrest detection and alerting device via wireless communication path 200 [b] look-up table in software to determine whether wearable cardiac arrest detection and alerting device is at any of the pre-programmed locations frequented by the particular individual being monitored by the wearable cardiac arrest detection and alerting device (e.g., locations such as individual's home, another home, office, fitness facility), [c] access to reverse geocoding data base to identify nearest phone numbers of potential first responders based on subject's GPS-derived location in the event the subject is not at one the frequented pre-programmed locations, [d] cellular phone communication component to call identified phone numbers of first responders identified above in [b] or [c] either the pre-programmed phone numbers if the subject is confirmed by reverse-geocoding to be at one of the including 911 (for use in the U.S.), and [e] audible synthesized speech to annunciate in placed phone calls that a cardiac arrest has occurred, identify the individual's name and specify the exact location of the individual in the form of subject's GPS location or equivalent device derived coordinates and, using reverse geocoding data base software, the actual address of the individual.

The range of dimensions for wearable cardiac arrest detection and alerting device 10 and accessory cellular phone and programmable device 39, as seen in FIGS. 2, 5, 6, 8, and 9 are summarized below in units of inches:
W1=0.75 to 1.50
W2=1.5 to 4.0
L1=0.75 to 2.00
L2=1.50 to 3.50
L3=3.0 to 6.0
t1=0.1 to 0.5

Alternatively, by way of example, but without limitation, the wearable apparatus and system of the present disclosure for the detection and alerting of first responders in the event of occurrence of a cardiac arrest or imminent cardiac arrest may be [a] a wearable cardiac arrest detection and alerting device in the form of a ring positioned on a finger of the hand, [b] a finger-tip mounted device, [c] a device mounted on the lower or upper arm, [d] a device mounted on the torso, [e] a device mounted on the forehead using a headband support, [f] a device mounted on an ear or [g] any other location on the body suitable for non-invasive, transcutaneous measurement of heart rate.

In yet another embodiment of the present disclosure, wearable sensors may be used to continuously monitor heart rate based on detectable electrical signals generated within the human body as a result of electrical impulses generated by the polarization and depolarization of cardiac tissue. The detectable electrical signals are the principle of widely used electrocardiography systems and methods. In this alternative embodiment, the detectable electrical signals are used to detect the wearer's heart rate in place of the photon sources and photodetectors based on the principle of photoplethysmography, as described with regard to FIGS. 1 through 6, 8, and 9. Except for the apparatus and method for detecting heart rate, the electrocardiography-based alternative embodiment of the present disclosure includes all the other components as specified in the foregoing disclosure associated with the photoplethysmography-based wearable cardiac arrest detection and alerting version of the present disclosure (in this regard, see Nemati, E. et. al, A Wireless Wearable ECG Sensor for Long-Term Applications. IEEE Communications Magazine 2012; 50 (1): 36-43), the latter reference incorporated herein by reference.

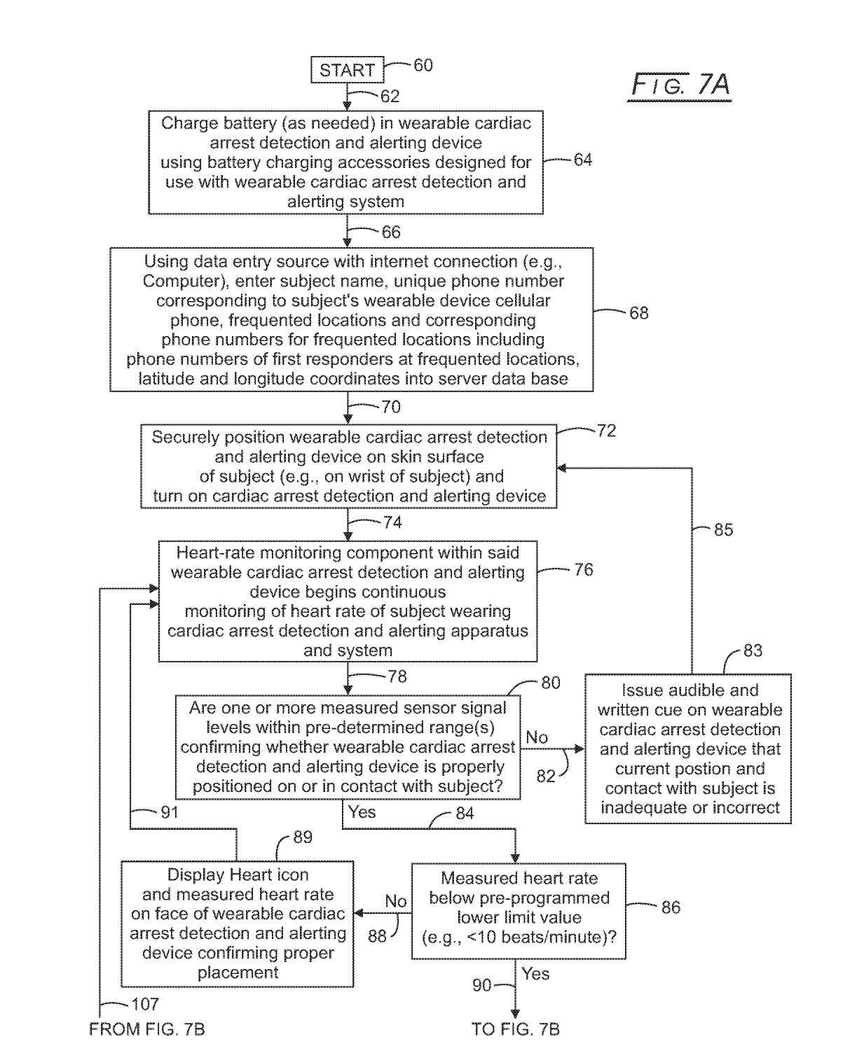

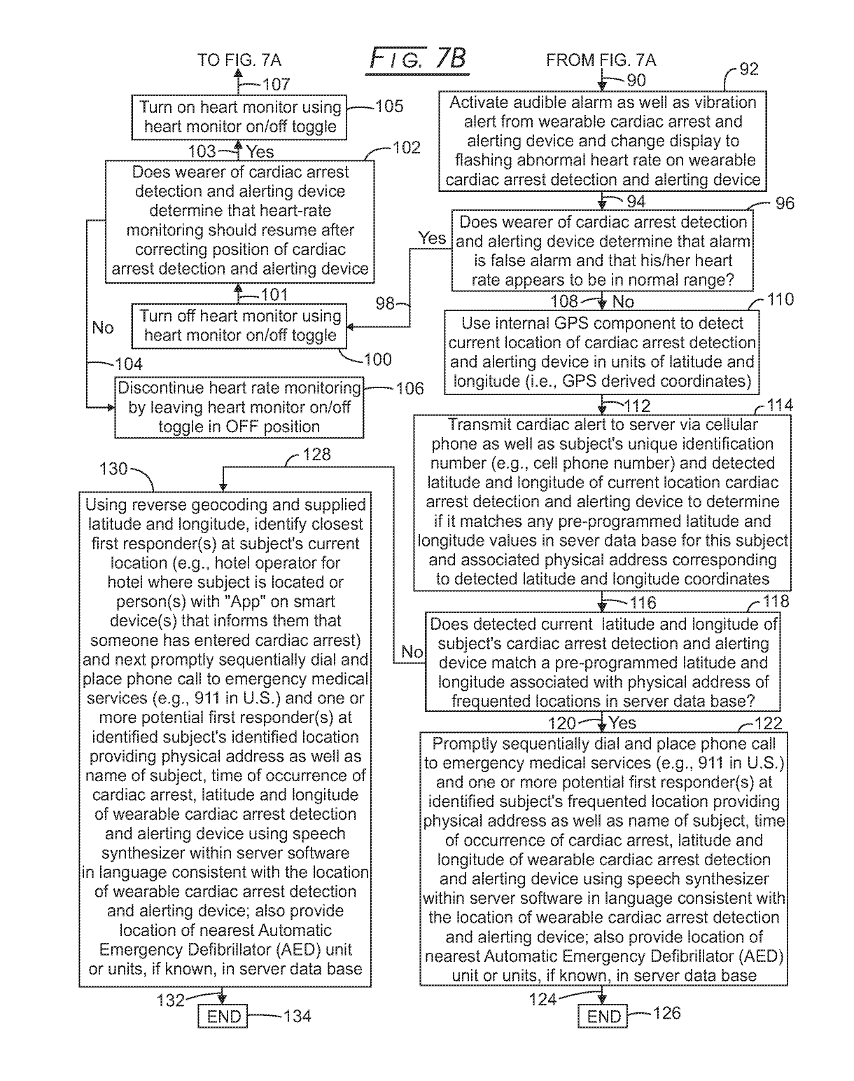

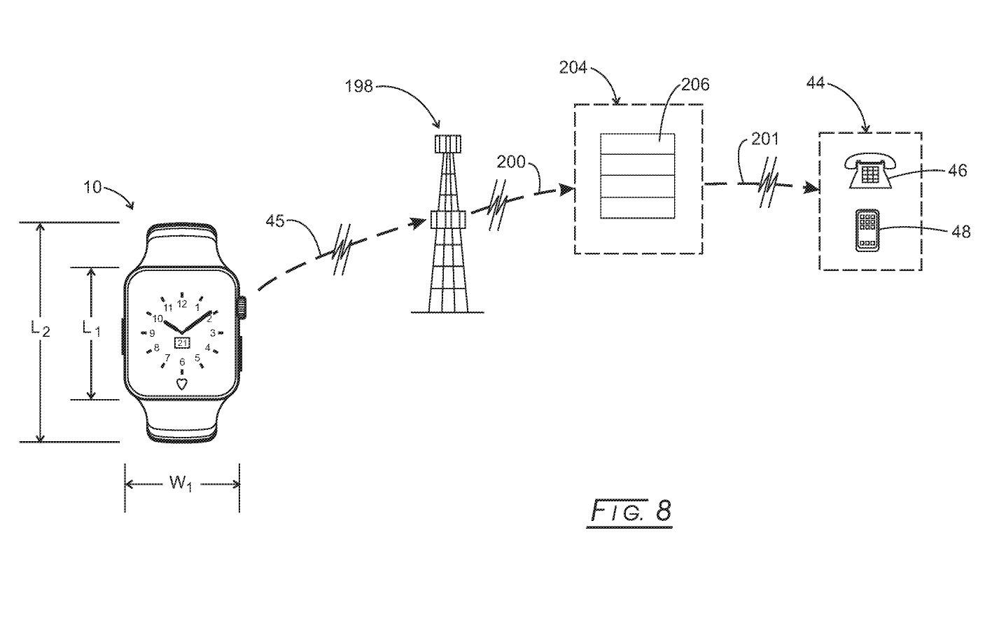

The operation and method of use of the wearable apparatus and system of a preferred embodiment of the present disclosure for the detection and alerting of first responders in the event of occurrence of a cardiac arrest or imminent cardiac arrest are set forth in the flow chart represented in FIGS. 7A and 7B in connection with FIGS. 1 through 4 and 8. Those figures should be considered as labeled thereon. Looking to FIG. 7A, the operation of wearable cardiac arrest detection and alerting device 10 commences with the charging of internal battery in wearable cardiac arrest detection and alerting device 10 as seen at arrow 62 and block 64. Once the required batteries are charged, data is entered into wearable cardiac arrest detection and alerting device 10 including the unique phone number of wearable cardiac arrest detection, and alerting device 10, identification (e.g., name) of wearer, addresses of frequently used locations and associated phone numbers into wearable cardiac arrest detection and alerting device 10, as seen at arrow 66 and block 68.

Next, wearable cardiac arrest detection and alerting device is securely positioned on skin surface of an individual and turned on to activate the cardiac arrest detection and alerting device, as seen at arrow 70 and block 72. Heart-rate monitor components within the wearable cardiac arrest detection and alerting device 10 begins continuous monitoring of heart rate of individual wearing cardiac arrest detection and alerting device, as seen at arrow 74 and block 76. By way of example, software within wearable cardiac arrest detection and alerting device 10 compares measured one or more sensor signals (e.g., optical signal level) with pre-determined range of one or more sensor signal levels (e.g., optical signal level) to determine whether wearable cardiac arrest detection and alerting device is properly positioned on individual, as seen at arrow 78 and block 80. If measured one or more sensor signals are not within range of pre-determined one or more sensor signal levels, then wearable cardiac arrest detection and alerting device issues audible and display cues as well as a vibration alert to individual being monitored indicating that wearable cardiac arrest detection and alerting device 10 is not properly positioned on individual, as seen at arrow 82 and block 83. As a consequence, the individual is alerted to securely position wearable cardiac arrest detection and alerting device 10, as seen at arrow 85 and block 72 and repeat subsequent steps leading to block 80.

Still referring to FIG. 7A and by way of example, if measured one or more sensor signal levels (e.g., optical signal level) are within range of pre-determined one or more sensor signal levels (e.g., optical signal level), then internal logic in wearable cardiac arrest detection and alerting device is used to determine whether measured heart rate is within normal pre-programmed physiological range to provide data necessary to determine cardiac arrest or imminent cardiac arrest has occurred, as seen at arrow 84 and block 86. If measured heart rate is greater than a pre-programmed physiological lower limit value (e.g., greater than or equal to 10 beats/minute), indicative that no cardiac arrest or imminent cardiac arrest has occurred, then wearable cardiac arrest detection and alerting device continues with monitoring of heart rate and display heart icon 20 and heart rate (see FIG. 1), as seen at arrow 88 and block 89, and proceeds with continuous monitoring of heart rate, as seen at arrow 91 and block 76.

Referring now to FIG. 7B and by way of example, if measured heart rate is less than a pre-programmed physiological lower limit value (e.g., less than 10 beats/minute), indicative that cardiac arrest has occurred or is imminent, then cardiac arrest or imminent cardiac arrest is determined to have occurred. As a result, the internal logic in wearable cardiac arrest detection and alerting device 10 [a] actuates audible alarm as well as a vibration alert and [b] changes display on the face of wearable cardiac arrest detection and alerting device 10 to flashing alert (e.g., “Cardiac Arrest”), as seen at arrow 90 and block 92. During a brief period of pre-programmed duration (e.g., say, 15 seconds) immediately following the start of the audible alarm as well as a vibration alert (referred to hereinafter as the “Alert Check Period”), the individual whose heart rate is being monitored has the opportunity to intervene, if the individual determines that their heart rate seems to be within a normal range and that a false alarm has occurred, as seen at arrow 94 and block 96. If the individual whose heart rate is being monitored determines that their heart rate seems to be within a normal range and that the alarm is a false alarm (e.g., due to unintended improper positioning of or contact with wearable cardiac arrest detection and alerting device), then the individual has the opportunity during the Alert Check Period to turn off wearable cardiac arrest detection and alerting device using heart monitor on/off toggle switch and next decides whether wearable cardiac arrest detection and alerting functions of wearable cardiac arrest detection and alerting device 10 should continue, as seen at arrow 98 and block 100.

If the individual whose heart rate is being monitored decides that wearable cardiac arrest detection and alerting device appears to be malfunctioning, then the individual turns off the wearable cardiac arrest detection and alerting device using heart monitor on/off toggle switch and discontinues its use, as seen at arrow 104 and block 106. Alternatively, if the individual whose heart rate is being monitored, decides that wearable cardiac arrest detection and alerting device appears to be functioning normally (e.g., after proper and secure repositioning of the wearable cardiac arrest detection and alerting device on body of individual wearing device), then the individual turns on the wearable cardiac arrest detection and alerting device using heart monitor on/off toggle switch, as seen at arrow 103 and block 105, and heart rate monitoring continues, as seen at arrow 107 and block 76 and as seen in FIGS. 7A and 7B.

Still referring to FIG. 7B, if the individual whose heart rate is being monitored decides that audible alarm, as well as a vibration alert, issued by wearable cardiac arrest detection and alerting device 10 appears to be a valid alarm or is unconscious or otherwise physically unable to turn off the wearable cardiac arrest detection and alerting device using heart monitor on/off toggle switch, then the audible alarm and procedure for alerting of first responders proceeds. At this time, with the audible alarm continuing, the internal GPS component within wearable cardiac arrest detection and alerting device 10 detects the location of wearable cardiac arrest detection and alerting device 10 in units of latitude and longitude coordinates, as seen at arrow 108 and block 110. Referring to FIGS. 8 and 7B, the detected latitude and longitude coordinate values of wearable cardiac arrest detection and alerting device 10 are transmitted by wearable cardiac arrest detection and alerting device 10 to server 206 using cellular phone communication. Server 206 compares transmitted latitude and longitude coordinate values of wearable cardiac arrest detection and alerting device 10 transmitted with pre-programmed latitude and longitude coordinate values in the database of server 206 to determine whether wearable cardiac arrest detection and alerting device 10 is at a pre-programmed physical address (e.g., home, office, fitness facility), as seen at arrow 112 and block 114. Software within server 206 determines whether wearable cardiac arrest detection and alerting device 10 is at a pre-programmed physical address, as seen at arrow 116 and block 118. If the detected latitude and longitude coordinate values of wearable cardiac arrest detection and alerting device 10 correspond to one of the pre-programmed pair of latitude and longitude coordinate values corresponding to physical address, then server 206 promptly issues phone calls to emergency phone number (e.g., 911 in the U.S.) and all other first responders associated with determined physical address and uses synthesized speech to identify name of individual, time of occurrence of cardiac arrest, physical address, as well as latitude and longitude coordinates, of wearable cardiac arrest detection and alerting device 10, as seen at arrow 120 and block 122.

Alternatively, as seen in FIG. 7B, if the detected pair of latitude and longitude coordinate values of wearable cardiac arrest detection and alerting device 10 do not correspond to one of the pre-programmed pair of latitude and longitude coordinate values corresponding to a physical address, then server 206 utilizes reverse geocoding in combination with latitude and longitude coordinate values transmitted by wearable cardiac arrest detection and alerting device 10 to promptly place telephone calls to emergency phone number (e.g., 911 in the U.S.), as well as one or more potential first responders, identified using reverse geocoding that are determined to be in close proximity to wearable cardiac arrest detection and alerting device 10 based on their respective latitude and longitude coordinate values (e.g., operator at hotel where individual is residing). Server 206 uses synthesized speech to identify name of individual, time of occurrence of cardiac arrest, and the physical address, as well as latitude and longitude coordinates, of wearable cardiac arrest detection and alerting device 10, as seen at arrow 128 and block 130.

In addition to issuing voice synthesized phone calls, text-based messages also can be issued by server 206 to emergency services (e.g., 911) and other first responders on the pre-programmed list wherein the other first responders contacted may be based on detected latitude and longitude coordinates of wearable cardiac arrest detection and alerting device 10. Also, the operation and method of use of the wearable apparatus and system of the present disclosure for the detection and alerting of first responders in the event of occurrence of a cardiac arrest or imminent cardiac arrest, as set forth in the flow chart represented in FIGS. 7A and 7B, also applies to the first, second, and fourth embodiment of the present disclosure for the detection and alerting of first responders in the event of a cardiac arrest, as illustrated pictorially in FIGS. 5, 6, and 9. As seen in FIG. 6, the apparatus and system of a second embodiment of the present disclosure includes a combination of both [a] wearable cardiac arrest detection and alerting device 10 and [b] accessory cellular phone and programmable device 39 maintained within the proximity of the wearable cardiac arrest detection and alerting device (e.g., cellular phone and programmable device 39 within 10 to 100 meters of wearable cardiac arrest detection and alerting device 10) during the period of monitoring. Hence, in the second embodiment of the present disclosure, some of the functions attributed solely to wearable cardiac arrest detection and alerting device 10, as presented in the foregoing description with regard to FIGS. 7A and 7B, are accomplished within the accessory cellular phone and programmable device 39, as seen in FIG. 6 and described in the description presented herein above.

Furthermore, the operation and method of use of the wearable apparatus and system of the present disclosure for the detection and alerting of first responders in the event of occurrence of a cardiac arrest or imminent cardiac arrest, as set forth in the flow chart represented in FIGS. 7A and 7B, also applies to other types of wearable cardiac arrest detection and alerting devices including [a] a wearable cardiac arrest detection and alerting device in the form of a ring positioned on a finger of the hand, [b] a finger-tip mounted device, [c] a device mounted on the lower or upper arm, [d] a device mounted on the torso, [e] a device mounted on the forehead using a headband support, [f] a device mounted on an ear, or [g] any other location on the body suitable for non-invasive, transcutaneous measurement of heart rate.

While the device and method have been described with reference to various embodiments, those skilled in the art will understand that various changes may be made and equivalents may be substituted for elements thereof without departing from the scope and essence of the disclosure. In addition, many modifications may be made to adapt a particular situation or material to the teachings of the disclosure without departing from the essential scope thereof. Therefore, it is intended that the disclosure not be limited to the particular embodiments disclosed, but that the disclosure will include all embodiments falling within the scope of the appended claims. In this application all units are in the metric system and all amounts and percentages are by weight, unless otherwise expressly indicated. Also, all citations referred herein are expressly incorporated herein by reference.

1. A wearable device for measuring one or more of heart rate or blood pressure of an individual whose skin is in contact with the wearable device and for communicating a cardiac arrest event, comprising:
[a] a first photon source having a wavelength in the range from 600 nm to 760 nm and directed at skin in contact therewith;
[b] a second photon source having a wavelength is in the range from 800 nm to 950 nm and directed at skin in contact therewith;
[c] one or more electro-optical photodetectors to receive photons from the skin that received photons from the first and second photon sources;
[d] a battery;
[e] a terminal assembly to enable charging of the battery;
[f] electrical components and an algorithm for converting the received photons into a measured of a wearer of the wearable device;
[g] digital memory to store a lower limit value of one or more of heart rate or blood pressure and identification indicia for the wearable device;
[h] an audible alarm to alert that the measured one or more of heart rate or blood pressure is less than the heart rate lower limit value;
[i] a global positioning satellite (GPS) receiver; and
[j] a wireless transmitter for transmitting to others:
[i] the identification indicia of the wearable device,
[ii] the GPS derived latitude and longitude coordinates of the wearable device, and
[iii] an alert that that the measured one or more of heart rate or blood pressure is less than the heart rate lower limit value indicative of a cardiac arrest event.

2. The wearable device of claim 1, wherein the heart rate lower limit value is 10 beats/minute.

3. The apparatus of claim 1, wherein the heart rate lower limit value is 2 beats/minute.

4. The wearable device of claim 1, wherein [a] first photon source wavelength is 560 nm and [b] second photon source wavelength is 577 nm.

5. The wearable device of claim 1, wherein an additional sensor detects level of contact of first and second photon sources as well as one or more electro-optical photodetectors.

6. The wearable device of claim 6, wherein sensor measures electrical impedance of adjacent skin surface of individual wearing device.

7. The wearable device of claim 6, wherein sensor measures temperature of adjacent surface of individual wearing device.

8. The wearable device of claim 6, wherein sensor measures electrical capacitance of surface of individual wearing device.

9. The wearable device of claim 6, wherein sensor measures pressure of contact between wearable device and of adjacent surface of individual wearing device.

10. The wearable device of claim 1, wherein on/off switch is accessible on wearable device to cancel alert in event wearable device not properly positioned on individual to enable measurement of heart rate.

11. The wearable device of claim 1, wherein the blood pressure lower limit is 10 mm Hg.

12. A system for measuring heart rate of individual and communicating an alert in event of a cardiac arrest comprising:
[a] a wearable device comprising:
[i] a heart rate measurement assembly attachable in monitoring relationship with individual wearing device,
[ii] a global positioning satellite (GPS) receiver,
[iii] a battery,
[iv] two terminals to enable charging of the battery in wearable device,
[v] an audible alarm to alert first responder(s) that measured heart rate is less than lower limit value,
[vi] digital memory to store identification of wearable device, and
[vii] a wireless transmitter for communication of identification of wearable device, GPS derived latitude and longitude coordinates of wearable device, and alert in the event of occurrence of heart rate less than lower limit value;
[b] a cellular phone communication receiver/transmitter located with range of wireless transmission of wearable device to relay wireless communication from wearable device to server;
[c] a server comprising hardware and software to:
[i] receive alert from wearable device,
[ii] translate information to be communicated in alert into synthesized speech including name of individual and physical location wearable device based on reverse geocoding of transmitted latitude and longitude coordinates,
[iii] contact emergency medical services via telephone or wireless cellular phone, and
[iv] contact one or more first responders via telephone and/or wireless cellular phone based on their proximity to wearable device.

13. The system of claim 12, additionally comprising an accessory cellular phone and programmable device that receives heart rate measured by wearable device via wireless communication with wearable device comprising a global positioning satellite (GPS) receiver, audible alarm to alert first responder(s) that measured heart rate is less than lower limit value, digital memory to store identification of wearable device, wireless transmitter for communication alert to server including identification of wearable device and GPS derived latitude and longitude coordinates.

14. A system for measuring heart rate of individual and communicating an alert in event of a cardiac arrest comprising:
[a] a cellular phone communication receiver/transmitter located with range of wireless transmission of wearable device to relay wireless communication from wearable device to one or more first responder; and
[b] a wearable device comprising:
[i] a heart rate measurement assembly attachable in monitoring relationship with individual wearing device,
[ii] a global positioning satellite (GPS) receiver,
[iii] a battery,
[iv] two terminals to enable charging of the battery in wearable device,
[v] an audible alarm to alert first responder(s) that measured heart rate is less than a lower limit value,
[vi] digital memory to store identification indicia of the wearable device,
[vii] a wireless transmitter for communication of identification indicia of the wearable device, GPS derived latitude and longitude coordinates of the wearable device, and an alert in the event of an occurrence of a heart rate less than the lower limit value, such alert including contacting emergency medical services and contacting one or more first responders, and
[viii] translation information to be communicated into synthesized speech including a name of individual and a physical location of the wearable device based on reverse geocoding of the transmitted latitude and longitude coordinates.

15. A method for detecting the occurrence of a cardiac arrest event, comprising the steps of:
providing a wearable heart rate detector;
providing a global satellite position receiver in the wearable heart rate detector;
providing digital storage of identification indicia of the individual wearing the wearable heart rate detector;
providing a sensor to detect adequacy of communication between the individual and the wearable heart rate detector to measure heart rate of the individual;
providing an audible alarm in the event that measured heart rate is less than a predetermined lower limit value;
providing wireless transmission to a server computer of an alert that a cardiac arrest has occurred in combination with identification indicia of the wearable heart rate detector, and latitude and longitude coordinates of the wearable heart rate detector;
utilizing forward and reverse geocoding by the server computer to identify the physical location of the wearable heart rate detector;
utilizing speech synthesis to translate an alert into audible information including the identification indicia of the individual wearing wearable heart rate detector, the physical location of wearable heart rate detector, and the time of occurrence of the cardiac arrest;
contacting emergency medical services via a wireless cellular phone transmitter; and contacting one or more first responders via the wireless cellular phone transmitter based on a proximity to the wearable heart rate detector.

16. A method for detecting the occurrence of a cardiac arrest, comprising the steps of:
providing a wearable heart rate detector;
providing a sensor to detect adequacy of communication between an individual and the wearable heart rate detector to enable heart rate measurement of the individual wearing the wearable heart rate detector;
providing an accessory cellular phone and programmable device to receive a measured heart rate from the wearable heart rate detector;
providing a global satellite position receiver within the accessory cellular phone and programmable device;
providing digital storage of identification indicia of the individual within the accessory cellular phone and programmable device;
providing wireless communication of the measured heart rate to the accessory cellular phone and programmable device;
providing an audible alarm in the event that the measured heart rate is less than a predetermined lower limit value;
providing wireless transmission from the accessory cellular phone and programmable device to a server computer hardware and software of an alert that cardiac arrest has occurred in combination with the identification indicia of the wearable heart rate detector, and latitude and longitude coordinates of the wearable heart rate detector;
utilizing forward and reverse geocoding by the computer server hardware and software to identify a physical location of the wearable heart rate detector;
utilizing speech synthesis by the server computer hardware and software to translate the alert into audible information including identification indicia of the individual wearing the wearable heart rate detector, a physical location of the wearable heart rate detector, and a time of occurrence of cardiac arrest;
contacting emergency medical services by the server computer hardware and software via a wireless cellular phone transmitter; and
contacting one or more first responders by the server computer hardware and software via telephone wireless cellular phone based on proximity of the one or more first responders to the wearable heart rate detector.

17. A wearable device for measuring heart rate of an individual and wirelessly communicating an alert and location of wearable device to one or more first responders in the event of a detected cardiac arrest.

18. A system comprising a wearable device that measures heart rate, global positioning satellite receiver, and wireless transmitter to communicate alert to a server that immediately communicates synthesized speech alert via telephone and/or cellular phone one or more first responders in the event of a detected cardiac arrest.

19. A system comprising a wearable device that measures heart rate, global positioning satellite receiver and wireless transmitter to communicate alert to an accessory cellular phone and programmable device that immediately communicates synthesized speech alert via cellular phone to one or more first responders in the event of a detected cardiac arrest.

20. A method for alerting one or more first responders in the event of a detected cardiac arrest comprising a wearable device for measuring heart rate of an individual wearing the wearable device and wireless communication via cellular phone to one or more first responders in the event of a detected cardiac arrest.

21. The method of claim 19, wherein the wireless communication is from the wearable device to an accessory cellular phone and programmable device that provides wireless communication to a server that issues an alert via a telephone and/or cellular phone to one or more first responders.

22. The method of claim 19, wherein the wireless communication is from the wearable device to server that issues alert via telephone and/or cellular phone to one or more first responders.

23. The method of claim 19, wherein the wireless communication is from the wearable device to an accessory cellular phone and programmable device that issues alert via cellular phone to one or more first responders.
